(19) United States
(12) Patent Application Publication
(10) Pub. No.: US 2006/0105432 A1
(43) Pub. Date: May 18, 2006

(54) Method for the production of vitamin b12

(75) Inventors: Heiko Barg, Speyer (DE); Dieter Jahn, Wolfenbuttel (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT

(21) Appl. No.: 10/541,920

(22) PCT Filed: Dec. 12, 2003
(86) PCT No.: PCT/EP03/14102

§ 371 Date: Aug. 5, 2005

(30) Foreign Application Priority Data
Jan. 11, 2003 (DE) 10300719.9

(51) Int. Cl. C12P 19/42; C12N 15/74; C12N 1/21

(52) U.S. Cl. 435/86; 435/471; 435/252.31

(57) ABSTRACT

The present invention relates to a method for the production of vitamin B12 by means of a culture comprising a genetically modified Bacillus megaterium strain, to a genetically modified Bacillus megaterium strain, and to vectors for its preparation.

The present invention a method for the production of vitamin B12 using a genetically modified Bacillus megaterium strain and vectors for the preparation of genetically modified bacteria of the genus Bacillus.

As a result of its effect on the human body, vitamin B₁₂ was discovered indirectly by George Minot and William Murphy as early as in the twenties of this century (Stryer, L., 1988, In Biochemie, fourth edition, pp. 528-531, Spektrum Akademischer Verlag GmbH, Heidelberg, Berlin, New York). Vitamin B₁₂ was purified and isolated first in 1948, and as little as eight years later, in 1956, Dorothy Hodgkin was successful in elucidating its complex three-dimensional crystal structure (Hodgkin, D. C. et al., 1956, Structure of Vitamin B₁₂. Nature 176, 325-328 and Nature 178, 64-70). The naturally occurring end products of vitamin B₁₂ biosynthesis are 5′-deoxyadenosylcobalamin (coenzyme B₁₂) and methylcobalamin (MeCbl), while vitamin B₁₂ is defined as cyanocobalamin (CNCbl), which constitutes the most frequently industrially produced and treated form. In the present invention, vitamin B₁₂, unless specified, uniformly stands for the name of all three analogous molecules.

The species B. megaterium was first described by De Bary as early as more than 100 years ago (in 1884). Although generally described as a soil-dwelling bacterium, B. megaterium can also be detected in various other habitats, such as salt water, sediments, rice, dried meat, milk or honey. The bacterium is frequently accompanied by pseudomonads and actinomycetes. Like its close relative Bacillus subtilis, B. megaterium is a Gram-positive bacterium and is distinguished, inter alia, by its relatively pronounced size of 2×5 μm, from which it obtains its name, a G+C content of approx. 38% and a highly pronounced ability to sporulate. Even very small amounts of manganese in the growth medium suffice for this species to perform a complete sporulation, an ability which is only comparable with the sporulation efficiency of some thermophilic Bacillus species. Owing to its size and its highly efficient sporulation and germination, a wide range of studies into the molecular bases of these methods were carried out on B. megaterium, so that, by now, more than 150 genes which are involved in its sporulation and germination are described for B. megaterium. Physiological studies on B. megaterium (Priest, F. G. et al., 1988, A Numerical Classification of the Genus Bacillus, J. Gen. Microbiol. 134, 1847-1882) classified this species as an obligate aerobic sporulating bacterium which is urease-positive and Voges-Proskauer-negative and not capable of reducing nitrate. One of the most outstanding characteristics of B. megaterium is its ability of utilizing a multiplicity of carbon sources. Thus, it utilizes a very high number of sugars and has been found, for example, in corn syrup, meat methoding waste and even in petrochemical waste. With a view to this ability of metabolizing a very broad spectrum of carbon sources, B. megaterium can be equated unreservedly with the pseudomonads (Vary, P. S., 1994, Microbiology, 40, 1001-1013, Prime time for Bacillus megaterium).

There is a wide range of advantages of broadly using B. megaterium in the industrial production of a very wide range of enzymes, vitamins and the like. One advantage is certainly the relatively highly developed genetics, which, within the genus Bacillus, is only exceeded by B. subtilis. Secondly, B. megaterium has no alkaline proteases, so that virtually no degradation was observed in the production of heterologous proteins. Moreover, it is known that B. megaterium efficiently secretes products of commercial interest, as is exploited for example in the case of the production of α- and β-amylase. Moreover, as a result of its size, B. megaterium is capable of accumulating a high biomass until an unduly high population density leads to its death. Most important in the industrial production by means of B. megaterium is furthermore the advantageous fact that this species is capable of producing products of high value and of very high quality from waste and inferior materials. This possibility of metabolizing an enormously wide substrate spectrum is also reflected in the use of B. megaterium as a soil detoxifying organism which is capable of degrading even cyanides, herbicides and persistent pesticides. Finally, the fact that B. megaterium is completely apathogenic and does not produce any toxins is of utmost importance, in particular in the production of foods and cosmetics. Because of these diverse advantages, B. megaterium is already being employed in a multiplicity of industrial applications, such as the production of α- and β-amylase, penicillin amidase, the methoding of toxic waste or the aerobic production of vitamin B₁₂ (for an overview, see Vary, P. S., 1994, Microbiology, 40, 1001-1013, Prime time for Bacillus megaterium).

Because of its many advantages in use in the biotechnological production of various products of industrial interest, the use of Bacillus megaterium is of great economic interest. Genetically optimized bacterial strains are increasingly being employed in order to increase the productivity of products of economic interest. However, genetically modified bacterial strains regularly entail problems regarding the stability of the freely replicable plasmids which they comprise. Moreover, a further improvement in the metabolite flux toward vitamin B12 and the directed control of the expression of chromasomally encoded genes during the bacterial fermentation are desirable for an optimal control of the product yield.

It is an object of the present invention to provide genetically modified Bacillus megaterium strains which allow the production of vitamin B12 to be further improved.

This furthermore requires the provision of suitable vectors which make possible an overexpression of the enzymes for the formation of uroporphyinogen-III from glutamyl-tRNA and, advantageously, a repression of the hem biosynthetic pathway together with an increased metabolite flux toward vitamin B12. At the same time, the vectors according to the invention should make possible the stable integration, into the chromosome of the bacterial strain, of the desired genetic modifications. Furthermore, an induction of the gene expression of the chromosomally encoded hemAXCDBL operon and/or the repression of the hem biosynthetic pathway during the fermentation should be controllable in a targeted manner.

The object is achieved by the provision of a genetically modified Bacillus megaterium strain comprising a gene hemA[KK] as shown in SEQ ID No. 4 coding for a feedback-resistant glutamyl-tRNA reductase and/or part of the nucleotide sequence as shown in SEQ ID No. 1 (hemZ) as antisense RNA (ashemZ).

A further embodiment of the present invention comprises a genetically modified Bacillus megaterium strain which comprises a gene hemA[KK] as shown in SEQ ID No. 4 coding for a feedback-resistant glutamyl-tRNA synthase, organized in a hemA[KK]XCDBL operon, and/or an antisense RNA (ashemZ) as shown in SEQ ID No. 3.

The present invention also comprises a nucleotide sequence as shown in SEQ ID No. 1, coding for a coproporphyrinogen-II oxidase.

This nucleotide sequence according to the invention is furthermore distinguished in that it comprises sequences with a regulatory function which precede (5′-, or upstream, sequences) and/or follow (3′-, or downstream, sequences) the region, of the hemZ gene, which codes for a coproporphyrinogen-III oxidase.

For the purposes of the invention, sequences with a regulatory function are understood as meaning those sequences which are capable of influencing transcription, RNA stability or RNA methoding, and translation. Examples of regulatory sequences are, inter alia, promoters, enhancers, operators, terminators or translation enhancers. However, this enumeration is not limiting for the present invention.

The nucleotide sequence according to the invention as shown in SEQ ID No. 1 is preferably derived from Bacillus megaterium. In this context, the present invention also relates to what are known as isolated nucleic acids. In accordance with the invention, an isolated nucleic acid, or isolated nucleic acid fragment, is understood as meaning an RNA or DNA polymer which can be single- or double-stranded and which may optionally comprise natural, chemically synthesized, modified or artificial nucleotides. In this context, the term DNA polymer also includes genomic DNA, cDNA or mixtures of these.

A coproporphyrinogen-III oxidase as shown in SEQ ID No. 2 is furthermore subject-matter of the present invention. The amino acid sequence as shown in SEQ ID No. 2 is preferably encoded by a nucleotide sequence as shown in SEQ ID No. 1. However, also encompassed in the present invention are alleles of the nucleotide sequence as shown in SEQ ID No. 1 coding for a coproporphyrinogen-III oxidase.

In accordance with the invention, alleles are understood as meaning functionally equivalent nucleotide sequences, i.e. nucleotide sequences which act essentially in the same sense. Functionally equivalent sequences are those sequences which, despite a deviating nucleotide sequence, for example as a result of degeneracy of the genetic code, still retain the desired functions. Thus, functional equivalents comprise naturally occurring variants of the sequences described herein, but also artificial nucleotide sequences, for example those which have been obtained by chemical synthesis and which have optionally been adapted to suit the codon usage of the host organism. Moreover, functionally equivalent sequences comprise those with a modified nucleotide sequence, which, for example, confers a desensitivity or resistance to inhibitors to the enzyme.

In principle, all the usual B. megaterium strains which are suitable as vitamin B12 production strains can be employed for the purposes of the present invention, i.e. for the generation of the genetically modified Bacillus megaterium strains.

For the purposes of the present invention, vitamin B12 production strains are to be understood as meaning Bacillus megaterium strains or homologous microorganisms which have been modified by traditional and/or molecular-genetic methods in such a way that their metabolite flux is increasingly directed toward the biosynthesis of vitamin B12 or its derivatives (metabolic engineering). In these production strains, for example one or more gene(s) and/or the corresponding enzymes at decisive key positions of the metabolic pathway (bottleneck), which, accordingly, are subject to complex regulation, are modified with regard to their regulation or indeed deregulated. In this context, the present invention comprises all of the known vitamin B12 production strains of the genus Bacillus or homologous organisms. The strains which are advantageous in accordance with the invention include in particular the strains of B. megaterium DSMZ32, DSMZ 509 and DSMZ 2894.

Bacterial strains which have been genetically modified in accordance with the invention can be generated, in principle, by traditional mutagenesis and, preferably, by directed molecular-biological techniques and suitable selection methods. Interesting approaches for the directed recombinant manipulation are, inter alia, branching sites of the biosynthetic pathways which lead to vitamin B12, by means of which the metabolite flux can be controlled in a targeted fashion toward a maximum vitamin B₁₂ production. Specific modifications of genes which are involved in the regulation of the metabolite flux also includes studies and modifications of the regulatory regions before and after the structural genes, such as, for example, the optimization and/or substitution of promoters, enhancers, terminators, ribosome binding sites and the like. Also comprised in accordance with the invention is the improvement of the stability of the DNA, mRNA or the proteins encoded thereby, for example by reducing or preventing degradation by nucleases or proteases, respectively.

In a variant of the present invention, the hemA[KK] gene as shown in SEQ ID No. 4 is integrated in the bacterial chromosome in the genetically modified Bacillus megaterium strain.

A further variant of a genetically modified Bacillus megaterium strain is distinguished by the fact that part of the hemZ gene is present as plasma-encoded antisense RNA (ashemZ) in an increased copy number in this bacterium.

For the purposes of the invention, part of the hemZ gene is understood as meaning that, starting from the nucleotide sequence of the hemZ gene as shown in SEQ ID No. 1, the preparation of various antisense RNAs possible. Procedures for the preparation of antisense RNA, for example via PCR, are known to the skilled worker and current laboratory practice. The differences can result for example from the length of the antisense RNAs which have been generated, or from the choice of the regions of the hemZ nucleotide sequence from which the antisense RNA(s) is/are derived. In this context, the antisense mRNA sequences can vary with regard to their length, for example between a few nucleotides and the entire sequence segment of the coding region. Preferred in accordance with the invention is an antisense RNA (ashemZ) as shown in SEQ ID No. 3.

The increased copy number can be the result of an increased replication of a suitable vector, resulting in an increased copy number.

In principle, an increased copy number can also be achieved by a multiple integration of a gene or parts thereof into the bacterial chromosome. Also comprised in accordance with the invention is a genetically modified Bacillus megaterium strain in which the hemA[KK] gene is integrated into the bacterial chromosome and part of the hemZ gene as antisense RNA (ashemZ) is present in an increased copy number.

Another subject-matter of the present invention is a genetically modified Bacillus megaterium strain in which the hemA[KK] gene, organized in the hemA[KK]XCDBL operon, and/or the part of the hemZ gene as antisense RNA (ashemZ) is under the control of an inducible promoter. Examples of inducible promoters are the xylose-inducible XylA promoter or the a beta-galactosidase-inducible promoter (Miller, J. H., 1972, Experiments in Molecular Genetics, Cold Spring Harbor Laboratory, Cold Spring Harbor, N.Y.). The xylose-inducible promoter which is preferred in accordance with the invention is the xylA promoter of the xylose operon from pWH1520 (Rygus, T. et al., 1991, Appl. Microbiol. Biotechnol., 35: 594-599). By adding xylose to the cultural medium, the initiation of the transcription of the genes under the control of the xylA promoter, that is to say in the present context the gene expression of hemA[KK]XCDBL and/or ashemZ, can be increased.

To prepare the above-described genetically modified Bacillus megaterium strains, vectors which are suitable in accordance with the invention and which are likewise subject-matter of the present invention are constructed.

Thus, the present invention comprises an integrative vector comprising a gene hemA[KK] coding for a feedback-resistant glutamyl-tRNA reductase as shown in SEQ ID No. 4 and, in operable linkage therewith, sequences for the induced gene expression, selection, replication and/or integration into the chromosome of the host cell.

An integrative vector is understood as meaning a vector which, owing to site-specific recombination, is integrated at a defined site into the host cell chromosome, where it replicates together with the chromosome. In a variant according to the invention, this site-specific recombination takes place via the homologous sequences of the hemA gene.

In accordance with the invention, homologous sequences are understood as meaning those sequences which are complementary to the nucleotide sequences according to the invention and/or hybridize therewith. In accordance with the invention, the term hybridizing sequences includes substantially similar nucleotide sequences from the group consisting of DNA or RNA which, under stringent conditions known per se, undergo a specific interaction (binding) with the abovementioned nucleotide sequences.

Starting from the DNA sequence described in SEQ ID NO: 4 or parts of these sequences, such homologous sequences can be isolated from other organisms, for example using customary hybridization methods or the PCR technique. These DNA sequences hybridize with the abovementioned sequences under standard conditions. It is advantageous to use short oligonucleotides, for example from the conserved regions, which can be determined in a manner with which the skilled worker is familiar via comparisons with other hemA genes in order to carry out the hybridization. However, it is also possible to use longer fragments of the nucleic acids according to the invention for the hybridization, or the complete sequences. Depending on the nucleic acid used: oligonucleotide, longer fragment or complete sequence, or depending on which type of nucleic acid, DNA or RNA, is being used for the hybridization, these standard conditions vary. Thus, for example, the melting points for DNA:DNA hybrids are approximately 10° C. lower than those of DNA:RNA hybrids with the same length.

Depending on the nucleic acid, standard conditions are understood as meaning, for example, temperatures of between 42° C. and 58° C. in an aqueous buffer solution with a concentration of between 0.1 to 5×SSC (1×SSC=0.15 M NaCl, 15 mM sodium citrate, pH 7.2) or additionally in the presence of 50% formamide (such as, for example, 42° C. in 5×SSC, 50% formamide). The hybridization conditions for DNA:DNA hybrids are advantageously 0.1×SSC and temperatures between approximately 20° C. to 45° C., preferably between approximately 30° C. to 45° C. For DNA:RNA hybrids, the hybridization conditions are advantageously 0.1×SSC and temperatures of between approximately 30° C. to 55° C., preferably between approximately 45° C. to 55° C. These abovementioned temperatures for the hybridization are examples of calculated melting point values for a nucleic acid with a length of approximately 100 nucleotides and a G+C content of 50% in the absence of formamide. The experimental conditions for the hybridization of DNA are described in relevant textbooks of genetics such as, for example, Sambrook et al., “Molecular Cloning”, Cold Spring Harbor Laboratory, 1989 and can be calculated using formulae with which the skilled worker is familiar, for example depending on the length of the nucleic acids, the type of the hybrids or the G+C content. The skilled worker can garner further information on the subject of hybridization from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

Furthermore, homologous sequences of the sequence mentioned in SEQ ID NO: 4 are understood as meaning for example variants which have at least 95% homology, preferably at least 96% homology, especially preferably at least 97 or 98% homology, very especially preferably at least 99 or 99.9% homology at the derived amino acid level. The homology was calculated over the entire amino acid region. The program PileUp was used (J. Mol. Evolution., 25, 351-360, 1987, Higgins et al., CABIOS, 5 1989: 151-153). For the purposes of the present invention, homology is understood as meaning identity. The two terms are synonymous.

An operable linkage is understood as meaning the sequential arrangement of, for example, promoter, coding sequence, terminator and, if appropriate, further regulatory elements in such a way that each of the regulatory elements can fulfill its intended function upon expression of the coding sequence. These regulatory nucleotide sequences can be of natural origin or can have been obtained by chemical synthesis.

A suitable promoter is, in principle, any promoter which is capable of controlling the expression of genes in the host organism in question. Preferred in accordance with the invention are chemically inducible promoters by means of which the expression of the genes which are subjected to them can be controlled at a particular point in time in the host cell. An example which may be mentioned here is the β-galactosidase-, arabinose- or xylose-inducible system. Preferred in accordance with the invention is the xylose-inducible system, and within this system the xylA promoter from pWH1520 (Rygus, T. et al., 1991, Appl. Microbiol. Biotechnol., 35: 594-599).

Thus, the invention also comprises an integrative vector of the above-described type where gene expression is under the control of the xylA promoter.

A large number of examples of sequences for the selection, replication and/or integration into the chromosome of the host cell are described in the literature. Thus, various selection markers are known, such as for example genes which confer a resistance to ampicillin, tetracyclin, kanamycin or erythromycin. However, this enumeration is not final or limiting for the present invention. Selection sequences which are advantageous according to the invention are the ampicillin resistance gene for selecting the vector in E. coli or the erythromycin resistance gene for its selection in B. megaterium.

Advantageous variants of an origin of replication in E. coli are pBR322 (Sutcliffe, J. G., 1979, Cold Spring Harbor Symp. Quant. Biol., 43, Pt 1: 77-90 or, in B. megaterium, pE194ts or repF. The temperature-sensitive origin pE194ts for B. megaterium only admits replication below 40° C., whereby a selection pressure can be built up above this “permissive” temperature for integration into the chromosone (Rygus et al., 1992). The repF gene product is described as an element which acts in-trans and which is required for the replication of the plasmid in Gram-positive bacteria (Villafane et al., 1987). A further variant of an integrative vector according to the invention is distinguished by comprising at least one temperature-sensitive origin of replication. An integrative vector comprising the temperature-sensitive origin of replication pE194ts is preferred.

A further variant of the present invention comprises an integrative vector which is distinguished in that it comprises a genetically modified nucleotide sequence of the hemA gene (hemA[KK]), which nucleotide sequence codes for a feedback-resistant glutamyl-tRNA synthase whose amino acid sequence comprises an insertion of at least two positively charged amino acids. The genetically modified nucleotide sequence which is present in the integrative vector preferably codes for a feedback-resistant glutamyl-tRNA which comprises 2 to 6, preferably 2 to 4 and especially preferably two additional amino acids. These additional amino acids can be introduced at the level of the nucleotide sequence coding for them by inserting two or, correspondingly, up to 6 triplets using procedures with which the skilled worker is familiar, for example via PCR, into the coding nucleotide sequence.

A preferred variant of the integrative vector comprises a genetically modified nucleotide sequence of the hemA gene (hemA[KK]), which nucleotide sequence codes for a feedback-resistant glutamyl-tRNA synthase whose amino acid sequence comprises, at positions 3 and 4 of the N terminus, an insertion of two positively charged amino acids. The positively charged amino acids are preferably lysine residues.

A feedback-resistant form of an enzyme is understood as meaning a protein whose activity is no longer inhibited by the end product of the metabolic pathway (or of a branch of the metabolic pathway). In accordance with the invention, this also comprises the enzyme of a feedback-resistant glutamyl-tRNA reductase with the amino acid sequence as shown in SEQ ID No. 5 encoded by the hemA[KK] gene from B. megaterium.

In this context, the genetically modified nucleotide sequence of the hemA gene (hemA[KK]) comprises naturally occurring variants of the hemA sequence described herein, but also an artificial nucleotide sequence, for example a nucleotide sequence obtained by chemical synthesis, which, if appropriate, has been adapted to suit the codon usage of the host organism. Genetic modifications comprise substitutions, additions, deletions, exchanges or insertions of one or more nucleotide residues.

Also included here are what are known as sense mutations, which, at the protein level, may for example lead to the substitution of conserved amino acids, but which do not lead to any basic change in the activity of the protein and are thus neutral with regard to function. Here, for example, certain amino acids can be replaced by amino acids with similar physico-chemical properties (spatial distribution, basicity, hydrophobicity and the like). For example, lysine residues are substituted for arginine residues, isoleucine residues for valin residues or glutamate residues for aspartate residues. This also comprises modifications of the nucleotide sequence which, at the protein level, affect the N or C terminus of a protein and which, while having no major adverse effect on the catalytic function of the protein, do indeed have a major adverse effect on the regulation of its activity. Indeed, these modifications can have a stabilizing effect on protein structure.

It is preferred to introduce, into the coding nucleotide sequence, 6 nucleotides which code for lysine while relying on the codon usage of B. megaterium. These modifications can be carried out by methods known per se.

Furthermore, the present invention comprises a vector comprising part of the nucleotide sequence as shown in SEQ ID No. 1 (hemz) as antisense RNA (ashemZ) and, in operable linkage therewith, sequences for the induced gene expression, selection, replication and/or integration into the chromosome of the host cell.

A preferred embodiment of the vector according to the invention comprises an antisense RNA (ashemZ) as shown in SEQ ID No. 3 and, in operable linkage therewith, sequences for the induced gene expression, selection, replication and/or integration into the chromosome of the host cell.

Preferred is an enhanced replication of the vector resulting in an increased copy number of part of the hemZ gene as antisense RNA (ashemZ), preferably of an antisense RNA (ashemZ) as shown in SEQ ID No. 3.

To obtain an increased gene expression (overexpression), it is possible to increase the copy number of the genes in question. Furthermore, the promoter and/or regulatory region and/or the ribosomal binding site, which is located upstream of the structural gene, can, correspondingly, be modified in such a way that expression takes place at an increased rate. Expression cassettes which are incorporated upstream of the structural gene can act analogously. By using inducible promoters, it is additionally possible to increase expression during the production of vitamin B12.

Measures for prolonging the lifespan of the mRNA likewise improve expression. The genes or gene constructs can either be present in plasmids in different copy numbers or else be integrated and amplified in the chromosome.

Furthermore, it is also possible for the activity of the enzyme itself to be elevated, for example to be increased by an elevated catalytic activity or a deregulated or feedback-desensitive (feedback-resistant) activity with regard to inhibitors or by the fact that the degradation of the enzyme protein is prevented. However, overexpression of the genes in question can furthermore be achieved by modifying the media composition and the culture procedure. In the host cell comprising part of the hemZ gene as antisense RNA (ashemZ), the resulting (expressed) antisense RNA anneals with the corresponding (complementary) region of the mRNA coding for coproporphyrinogen-III oxidase.

Preferably, it thereby blocks the ribosomal binding site of the hemZ gene, thus inhibiting the translation and expression of the key enzyme which is involved in hem biosynthesis. This, in turn, results in a reduced hem biosynthesis, with the advantage of an increased flux of metabolic metabolites toward the production of vitamin B12.

The present invention furthermore relates to a vector comprising part of the nucleotide sequence as shown in SEQ ID No. 1 (hemZ) as antisense RNA (ashemZ), preferably of an antisense RNA (ashemZ) as shown in SEQ ID No. 3, where gene expression is under the control of the xylA promoter.

In principle, this vector is furthermore also capable of integrating into the chromosome of the host cell, for example when equipped with a temperature-sensitive origin of replication. One variant of this vector comprises at least one temperature-sensitive origin of replication. Preferably, such a vector variant comprises the temperature-sensitive origin of replication pE194ts.

The vectors according to the invention are prepared by fusing the abovementioned components, such as promoter, coding gene segments, origin of replication, genes for selection, or the like, using customary recombination and cloning techniques as are described for example in Sambrook, J. et al., 1989, In Molecular cloning; a laboratory manual. 2^{nd} Ed., Cold Spring Harbor Laboratory Press, Cold Spring Harbor, N.Y. Adapters or linkers may be added to the fragments for linking the DNA fragments with one another.

The present invention furthermore relates to the use of the integrative vector comprising the hemA[KK] gene of the above-described type for the preparation of a Bacillus megaterium strain which has been genetically modified in accordance with the invention.

Likewise, the present invention comprises the use of a nucleotide sequence as shown in SEQ ID No. 1 for the preparation of an antisense RNA (ashemZ) as shown in SEQ ID No. 3. Furthermore, the present invention comprises the use of an antisense RNA (ashemZ) as shown in SEQ ID No. 3 for the preparation of a vector of the abovementioned type comprising part of the hemZ gene as shown in SEQ ID No. 1 as antisense RNA (ashemZ) as shown in SEQ ID No. 3. The present invention furthermore relates to the use of a vector comprising part of the hemZ gene as shown in SEQ ID No. 1 as antisense RNA (ashemZ) as shown in SEQ ID No. 3 for the preparation of a genetically modified Bacillus megaterium strain of the type according to the invention. In accordance with the invention, it is also possible to transfer the integrative vector comprising the hemA[KK] gene and the vector comprising an antisense RNA (ashemZ) into a suitable Bacillus megaterium strain and to employ the resulting genetically modified strain for the production of vitamin B12.

The present invention thus also relates to the use of a genetically modified Bacillus megaterium strain of the described type for the production of vitamin B12.

The present invention furthermore relates to a method for the production of vitamin B12 by means of a culture comprising a genetically modified Bacillus megaterium strain of the described type, the fermentation being carried out under aerobic conditions.

In one variant of the method according to the invention, in the exponential growth phase of the aerobically fermenting cells, a transition from aerobic to anaerobic fermentation conditions takes place. The vitamin B12 production can be increased even further by means of this step, which is known as a shift, or by a two-step fermentation method.

Advantageous in accordance with the invention in this context is a method in which the transition from the aerobic to the anaerobic fermentation takes place as soon as the aerobic culture has reached its maximum optical density, but at least an optical density of approximately 2 to 3. As a rule, the optical density is determined at 570-600 nm.

Anaerobic conditions for the purposes of the present invention are understood as meaning those conditions which prevail when the bacteria are first grown aerobically and then transferred into anaerobic bottles, where they are fermented. The time of transfer into the anaerobic bottles takes place, in particular in the two-stage method, as soon as the aerobically grown bacterial cells are in the exponential growth phase. This means that, after transfer into the anaerobic bottles, the bacteria consume the oxygen which is present therein, and no further oxygen is supplied. These conditions may also be referred to as semi-anaerobic. The corresponding procedures are conventional laboratory practice and known to the skilled worker.

Comparable conditions also prevail when the bacteria are first grown aerobically in a fermenter and then the oxygen supply is gradually reduced so that semi-anaerobic conditions are eventually established. As an alternative, the oxygen may also be expelled actively via passing in inert gas, such as nitrogen.

In a special variant of the present invention, it is also possible for example to create strictly anaerobic conditions by adding reducing agents to the culture medium.

In general, it is not absolutely necessary for a fermentation of the invention under anaerobic conditions (whether semi-anaerobic or strictly anaerobic) for the bacteria to be grown aerobically (preculture). This means that the bacteria can also be grown under anaerobic conditions and then be fermented further under semi-anaerobic or strictly anaerobic conditions. It is also conceivable for the inoculum to be taken directly from strain maintenance and employed for the production of vitamin B12 under anaerobic conditions.

The Bacillus megaterium strain which has been genetically modified in accordance with the invention can also be fermented in a batch culture. Variants which are fermented in a fed-batch culture or continuously are also encompassed by the invention.

Advantageous in accordance with the invention is a method in which the expression of the hemA[KK]XCDBL operon and/or the expression of the nucleotide sequence which codes for an antisense RNA of the hemZ gene (ashemZ) is induced by the addition of xylose to the fermentation medium. The present invention also relates to a variant of the abovementioned method for the production of vitamin B12 in which the expression of the hemA[KK]XCDBL operon as shown in SEQ ID No. 4 and/or the expression of the nucleotide sequence which codes for an antisense RNA of the hemZ gene (ashemZ) as shown in SEQ ID No. 3 is induced by the addition of xylose to the fermentation medium.

In methods according to the invention for the production of vitamin B12, xylose concentrations of approximately 0.1 to 1% prove to be advantageous. An addition of approximately 0.2 to 0.5% xylose to the culture medium is preferred. Especially preferred is the addition of approximately 0.20-0.25%, in particular 0.23%, of xylose under aerobic fermentation conditions and of 0.4-0.5%, in particular 0.5%, under anaerobic fermentation conditions.

The overexpression according to the invention of the hemA[KK]XCDBL operon in the genetically modified B. megaterium strain which comprises the hemA[KK]XCDBL operon integrated in the chromosome under the induced control of the xylA promoter (“integrated strain”) leads to an increase in the vitamin B₁₂ content by a factor of at least 15-40, preferably 20-35, especially preferably 22 on comparing the B. megaterium strain DSMZ509 with the “integrated” strain (μg/l×OD). When calculating the increase in the vitamin B12 production in μg/l, an increase by a factor of at least 15-40 results, preferably 20-35, especially preferably 30.

The overexpression according to the invention of the ashemZ gene in a genetically modified B. megaterium strain, such as, for example, DSMZ509, which is based for example as the result of an increased copy number in the cell and which can additionally be induced by adding xylose to the culture medium leads, at the point in time of approximately 3 hours post-induction with xylose, to a vitamin B12 content which is increased by the factor of approximately 15-40%, preferably 20-35%, especially preferably 22% over the comparative strain. At the point in time of approximately 6 hours post-induction with xylose, for example, an increase in the vitamin B12 content of approximately 16% may be present.

A comparative strain is understood as meaning a B. megaterium strain which likewise harbors a vector, but without ashemZ insert.

In one variant of the method according to the invention, at least cobalt and/or 5-aminolavulic acid is/are added to the culture medium.

The fermentation is advantageously carried out under aerobic conditions with addition of approximately 250 μM cobalt; under anaerobic conditions, an addition of up to 500 μM cobalt is advantageous. When adding 5-aminolavulic acid, up to 300 μM are advantageous under aerobic and anaerobic conditions. In a variant of the method according to the invention, the vitamin B12 content can be raised by the addition of approximately 200 to 750 μM, preferably 250 to 500 μM, of cobalt per liter of culture medium.

In the case of growth with cobalt and ALA, at least 1-25%, preferably 5-18% and especially preferably 10% more vitamin B₁₂ are formed six hours post-induction with xylose in the genetically modified B. megaterium DSMZ509-pHBasHemZ than in the comparative strain.

This shows that the transcription of the antisense hemZ RNA not only inhibits the synthesis of hems, but simultaneously leads to an increased vitamin B₁₂ formation. Inhibition of the hem synthesis increasingly directs the metabolite flux of tetrapyrrole synthesis toward the vitamin B₁₂ synthetic pathway.

After the fermentation, the vitamin B12 which has been formed can be methoded from the fermentation medium. Such measures are conventional laboratory practice and will not be described in further detail here.

The present invention is illustrated in greater detail by the examples which follow, but which should not be construed as limiting:

Bacterial Strains and Plasmids

Bacterial strains and plasmids as shown in Tables 1 and 2 hereinbelow were employed.

TABLE 1
Bacterial strains used

| Strain | Description | Reference/source |
|---|---|---|
| Escherichia coli | F⁻mcrA Δ(mrr-hsdRMS-mcrBC) | Gibco ® BRL |
| DH10B | φ80dlacZΔM15 ΔlacX74 deoR | |
| | recA1 endA1 araD139 Δ(ara, | |
| | leu)7697 galU galK λ-rpsL nupG | |
| Bacillus | Vitamin B₁₂ producer | DSMZ* |
| megaterium | | |
| DSMZ509 | | |

*DSMZ: Deutsche Sammlung von Mikroorganismen [German Collection of Microorganisms], Brunswick

TABLE 2
Plasmids used

| Plasmid | Description | Reference/source |
|---|---|---|
| pWH1520 | Cloning and expression vector | Rygus et al., 1991 |
| | for Bacillus spp., | |
| | Ap^{r}, Tc^{r} | |
| pHBasHemZ | 129 bp antisense RNA against B. | present work |
| | megaterium hemZ gene in | |
| | pWH1520 | |
| pWH1967E | Cloning, expression and integration | Schmiedel, D. et |
| | vector for Bacillus spp., Ap^{r}, | al., 1997 |
| | Tc^{r}, Ery^{r} | |
| pMM1520 | pWH1520 with MCS | Malten, M., 2002 |
| pHBintE | Cloning, expression and integration | present work |
| | vector for Bacillus spp., | |
| | Ap^{r}, Ery^{r}, ori pE194 ts | |
| pHBiHemAKK | Integration vector for Bacillus spp. | present work |
| | with a HemA mutant, Ap^{r}, Ery^{r}, | |
| | ori pE194 ts | |

Buffers and solutions
Minimal media

| Mopso minimal medium | | |
|---|---|---|
| Mopso(pH 7.0) | 50.0 | mM |
| Tricine(pH 7.0) | 5.0 | mM |
| MgCl₂ | 520.0 | μM |
| K₂SO₄ | 276.0 | μM |
| FeSO₄ | 50.0 | μM |
| CaCl₂ | 1.0 | mM |
| MnCl₂ | 100.0 | μM |
| NaCl | 50.0 | mM |
| KCl | 10.0 | mM |
| K₂HPO₄ | 1.3 | mM |
| (NH₄)₆Mo₇O₂₄ | 30.0 | pM |
| H₃BO₃ | 4.0 | nM |
| CoCl₂ | 300.0 | pM |
| CuSO₄ | 100.0 | pM |
| ZnSO₄ | 100.0 | pM |
| D-Glucose | 20.2 | mM |
| NH₄Cl | 37.4 | mM |

Titration reagent was KOH solution.
15 g/l agar-agar were added for solid media.

Solutions for protoplast transformation of Bacillus megaterium

| | | |
|---|---|---|
| SMMP buffer | | |
| Antibiotic Medium No. 3 (Difco) | 17.5 | g/l |
| Sucrose | 500.0 | mM |
| Na maleate (pH 6.5) | 20.0 | mM |
| MgCl₂ | 20.0 | mM |
| PEG-P solution | | |
| PEG 6000 | 40.0% | (w/v) |
| Sucrose | 500.0 | mM |
| Na maleate (pH 6.5) | 20.0 | mM |
| MgCl₂ | 20.0 | mM |
| cR5 top agar | | |
| Sucrose | 300.0 | mM |
| Mops (pH 7.3) | 31.1 | mM |
| NaOH | 15.0 | mM |
| L-Proline | 52.1 | mM |
| D-Glucose | 50.5 | mM |
| K₂SO₄ | 1.3 | mM |
| MgCl₂ × 6 H₂O | 45.3 | mM |
| KH₂PO₄ | 313.0 | μM |
| CaCl₂ | 13.8 | mM |
| Agar-agar | 4.0% | (w/v) |
| Casamino acids | 0.2% | (w/v) |
| Yeast extract | 10.0% | (w/v) |

Titration reagent was NaOH solution.

Media and Additions to Media

Unless stated specially, the Luria-Bertani Broth (LB) complete medium as described in Sambrook et. al (1989) was used. 15 g of agar were additionally added per liter of solid media.

Additions

Additions such as carbon sources, amino acids, antibiotics or salts were either added to the media and autoclaved together or made up as concentrated stock solutions in water and sterilized or filter-sterilized. The substances were added to the media which had been autoclaved and cooled to below 50° C. In the case of substances which are sensitive to light, such as tetracyclin, care was taken to incubate in the dark. The final concentrations normally used were the following; however, this does not mean that variations are not possible:

| | | |
|---|---|---|
| ALA | 298 | μM |
| Ampicillin (for E. coli) | 296 | μM |
| CoCl₂ (in aerobic cultures) | 250 | μM |
| Erythromycin (for B. megaterium) | 0.55 | μM |
| | 102 | μM |
| Glucose | 22 | mM |
| Lysosyme | 1 | mg/ml |
| Tetracyclin (in solid media) | 23 | μM |
| Tetracyclin (in liquid media) | 68 | μM |
| Xylose | 33 | mM |

Microbiological Techniques
Sterilization

Unless indicated otherwise, all the media and buffers were steam-sterilized for 20 minutes at 120° C. and a gage pressure of 1 bar. Heat-sensitive substances were filter-sterilized (pore diameter of the filter 0.2 μm), and glassware was heat-sterilized at 180° C. for at least 3 hours.

General Growth Conditions for Liquid Bacterial Cultures

Using a sterile loop, bacteria were removed from an LB agar plate or from a glycerol culture and inoculated into the nutrient medium which, if required, comprised an antibiotic.

Aerobic bacterial cultures were incubated in baffle flasks at 37° C. at a speed of 180 rpm. The incubation times varied according to the desired optical densities of the bacterial cultures.

Growth Conditions for Bacillus megaterium

For the best possible aeration of aerobic cultures, these were incubated in baffle flasks at 250 rpm and 37° C. Anaerobic cultures were grown in a volume of 150 ml in 150-ml anaerobic bottles at 37° C. and 100 rpm. In both cases, care was taken to inoculate in the ratio 1:100 from overnight cultures, and to use constant conditions for the overnight cultures. In order to achieve higher cell biomass yields under anaerobic conditions, B. megaterium cultures were preincubated aerobically and, when the density had reached the desired value, switched to anaerobic growth conditions. To this end, B. megaterium was first incubated in baffle flasks at 250 rpm and 37° C. In the middle of the exponential growth, or at the beginning of the stationary phase, all of the culture was transferred into a 150-ml anaerobic flask and grown on at 37° C. and 100 rpm.

Bacterial Plate Cultures

Using a sterile loop, bacteria were removed from a glycerol culture and fractionally streaked onto an LB agar plate which, if required, had been treated with a suitable antibiotic, so that, following incubation overnight at 37° C., single colonies were discernible on the plate. If bacteria from a liquid culture were used, they were streaked on the LB agar plate using a Drygalski spatula and then incubated overnight at 37° C.

Determination of the Cell Density

The cell density of a bacterial culture was determined by measuring the optical density (OD) at 578 nm, the assumption being that an OD₅₇₈ of one corresponds to a cell count of 1×10⁹ cells.

Storage of Bacteria

The long-term storage of bacteria involved what are known as glycerol cultures. To this end, 850 μl of a bacterial overnight culture were mixed thoroughly with 150 μl of sterile 85% glycerol and the mixture was stored thereafter at −80° C.

Molecular Biology Methods

The standard work for the above-described molecular biology methods is Sambrook et al. (1989).

Preparation of Competent Cells

To prepare competent E. coli cells, 500 ml of liquid cultures were grown with LB medium to an OD₅₇₈ of 0.5-1. The culture was cooled on ice and then centrifuged (4000×g; 15 min; 4° C.). The cell sediment was resuspended thoroughly in sterile deionized water, centrifuged (4000×g; 8 min; 4° C.), again washed with sterile deionized water and again centrifuged (4000×g; 8 min; 4° C.). After the sediment had been washed with 10% (v/v) glycerol solution, the mixture was centrifuged (4000×g; 8 min; 4° C.) and the sediment was resuspended in as little as possible 10% (v/v) glycerol solution. The competent E. coli cells were immediately used for the transformation or else frozen at −80° C.

Transformation of Bacteria by Electroporation

The transformation was carried out by means of electroporation with the aid of a Gene Pulser with attached Pulse Controller (BioRad). To this end, in each case 40 μl of competent E. coli cells and 1 μg of plasmid DNA were transferred into a transformation cuvette and, in the Gene Pulser, exposed to a field strength of 12 kV/cm at 25 pF and a parallel resistance of 200 Ω. In the event that more than 2 μl of the plasmid DNA have to be added, a dialysis was carried out.

For the subsequent regeneration, the transformed cells were, immediately after the transformation, incubated in 1 ml of LB medium in a thermoshaker at 37° C. for half an hour. Thereafter, various volumes of these batches were scraped onto LB plates with appropriate addition of antibiotics and incubated overnight at 37° C.

Protoplast Transformation of Bacillus megaterium

Protoplast Preparation

50 ml of LB medium were inoculated with 1 ml of an overnight culture of B. megaterium and incubated at 37° C. At an OD₅₇₈ of 1, the cells were centrifuged (10 000×g; 15 min; 4° C.) and resuspended in 5 ml of freshly prepared SMMP buffer. After addition of lysosyme in SMMP buffer, the suspension was incubated for 60 minutes at 37° C., and the formation of protoplasts was monitored under the microscope. The cells were harvested by centrifugation (3000×g; 8 min; Rt) and the cell sediment was then carefully resuspended in 5 ml of SMMP buffer, and the centrifugation and washing steps were carried out for a second time. It was then possible, after adding 10% (v/v) glycerol, to divide the protoplast suspension into portions and freeze them at −80° C.

Transformation

500 μl of the protoplast suspension were treated with 0.5 to 1 μg of DNA in SMMP buffer, and 1.5 ml of PEG-P solution were added. After incubation at Rt for 2 minutes, 5 ml of SMMP buffer were added and carefully mixed, and the suspension was centrifuged (3000×g; 5 min; RT). Immediately thereafter, the supernatant was removed, and the sediment, which was barely visible, was resuspended in 500 μl of SMMP buffer. The suspension was incubated for 90 minutes at 37° C. with gentle shaking. Thereafter, 50-200 μl of the transformed cells were mixed with 2.5 ml of cR5 Top agar and put onto LB-agar plates which comprised the antibiotics suitable for selection. Transformed colonies were discernible after incubation at 37° C. for one day.

Cloning and Sequencing the hemZ Gene from Bacillus megaterium

To sequence the hemZ gene from Bacillus megaterium DSMZ509, genomic DNA was isolated and employed as template in a PCR reaction with the following primers:

| | |
|---|---|
| PCR primer 1: | 5′-TTTATATTCATATTCCATTTTG-3′ |
| PCR primer 2: | 5′-GGTAATCCAAAAATAAAATC-3′ |

A 480 bp PCR fragment with 65.1% identity with the hemZ gene from B. subtilis which constitutes a part-sequence of the hemZ gene from B. megaterium was amplified. To complement the hemZ part-sequence, a unidirectional PCR, i.e. what is known as vectorette PCR, was carried out using the vectorette system from Sigma Geneosys.

The vectorette PCR allows the amplification of unknown DNA regions which border known sequence segments. Here, a first primer is designed on the basis of the known DNA sequence. To establish a known DNA sequence for the hybridization of the second PCR primer required, the genome is cut using a restriction endonuclease, and all the resulting ends are linked with a known short DNA sequence. After the synthesis of the primary strand, this short sequence (vectorette) acts as target sequence for the second primer. All of the restriction digest fused with the vectorette units can be regarded as a sort of gene library, the vectorette library, with the aid of which any desired sequence can be amplified. Since the vectorette consists of an oligonucleotide double strand, parts of which are not paired, the complementary PCR primer for the second amplification cycle can only hybridize when the primer which is specific for the known sequence region has been elongated and given rise to the complementary sequence. This ensures that only the desired DNA is amplified.

A successful vectorette PCR requires a fragment size, of the desired genomic DNA, which is capable of being amplified. Here, the fragment size should not exceed 6-7 kb so that a specific DNA polymerase (Taq) can synthesize the fragment up to the end, without disruption. An adequate restriction enzyme for the digestion of the genomic DNA is determined by a preliminary Southern Blot analysis. The restriction enzyme Clal was chosen for this purpose. The fragment size which has been determined by the Southern Blot analysis permits the calculation of the size of the PCR fragment to be expected and thus facilitates its identification.

The vectorette PCR resulted in the isolation of one strand of the complete hemZ gene from Bacillus megaterium. Starting from this sequence, it was possible to amplify and sequence all of the hemZ gene in its entirety, using inverse PCR. The sequence is shown in SEQ ID No. 1.

Vector Constructions

Construction of pHBintE

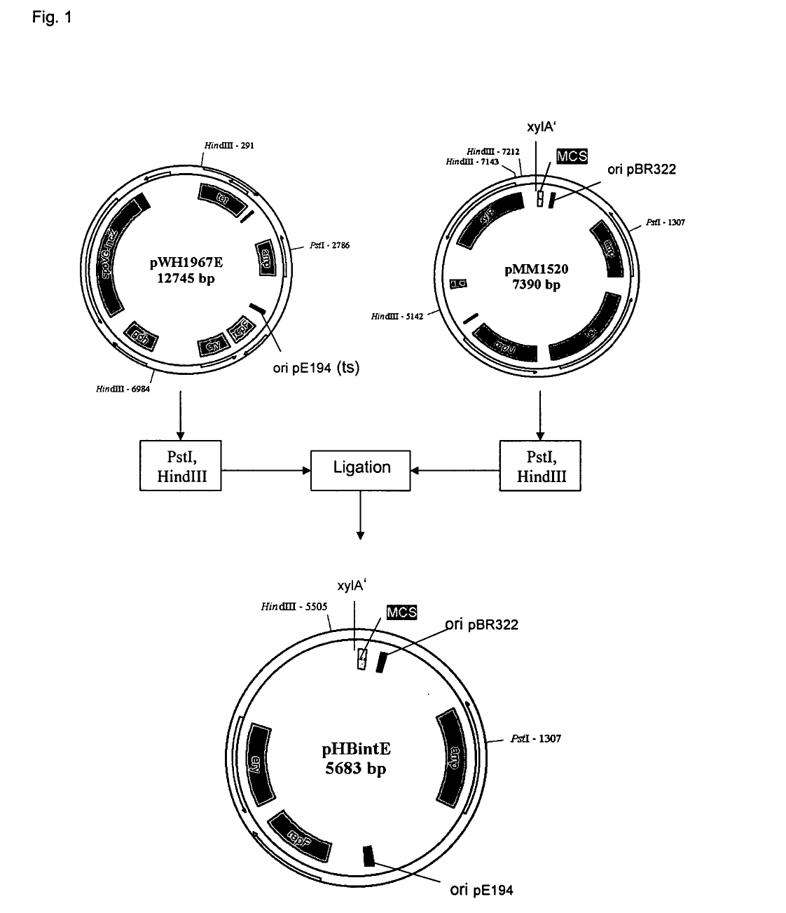

The starting plasmids used were pWH1967E (Schmiedel, D. et al., 1997, Appl. Microbiol. Biotechnol., 47 (5): 543-546) and pMM1520 (Malten, Marco, 2002, Produktion und Sekretion einer Dextransucrase in Bacillus megaterium [Production and Secretion of a Dextran Sucrase in Bacillus megaterium], PhD thesis, Institute of Microbiology (Prof. Dr. D. Jahn), Technical University Brunswick). First, the two plasmids were cut with in each case PstI and HindIII. Thereafter, the complete mixtures were each applied to one agarose gel, and the fragments of interest were eluted. The eluted fragment of pWH1967E (4198 bp) comprised an erythromycin resistance, the repF gene, the temperature-sensitive origin pE194ts and half an ampicillin resistance. The fragment of pMM1520 (1485 bp) comprised the xylA′ promoter from B. megaterium, and, directly upstream of the promoter, a multiple cloning site, the origin from pBR322 and the second part of the ampicillin resistance gene, which complements the ampicillin resistance. The cohesive ends of the two fragments were then ligated. The resultant plasmid was named pHBintE. The cloning strategy is shown schematically in FIG. 1.

Thus, the cloned plasmid pHBintE (FIG. 1) has the following characteristics. It has an ampicillin resistance for selection in E. coli transformants and an erythromycin resistance for the selection of B. megaterium transformants. The important elements for the replication in E. coli (pBR322) and B. megaterium (pE194ts and repF) are present. The temperature-sensitive origin pE194 ts for B. megaterium only permits replication below 40° C., whereby it is possible to build up, above this “permissive” temperature, a selection pressure for integration into the chromosome (Rygus et al, 1992). The repF gene product is described as an element which acts in trans and which is required for the replication of the plasmid in Gram-positive bacteria (Villafane et al., 1987). Moreover, the plasmid comprises the xylA′ promoter with a multiple cloning site directly upstream. This promoter makes possible the induction, by means of xylose, of genes inserted into the multiple cloning site.

Construction of pHBiHemA[KK]

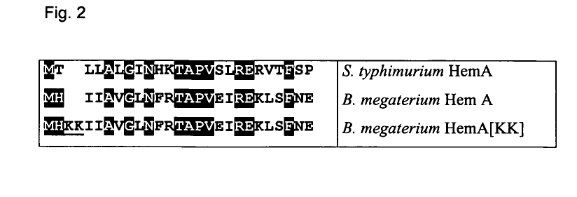

FIG. 2 shows the first 27 amino acids of the alignment report for HemA of B. megaterium with “KK-deregulated HemA”, B. megaterium wild type and of S. typhimurium. This figure shows again clearly the site at which the insertion is to take place.

The HemA[KK] mutant was cloned by means of PCR. The template used was chromosomal DNA from B. megaterium. Since the sequence of the hemA gene of B. megaterium was known, it was possible to derive primers. The sequences of the primers are shown hereinbelow:

| | |
|---|---|
| Forward | 5′ GGGGACTAGTCAAATGCATAAAAAAATTATAGCAGTC |
| | GG 3′ |
| Reverse | 5′ CTGGGGTACCCCATATCAACCATTATTCAATCC 3′ |

The derived primers lacked complete homology with the B. megaterium sequence. Firstly, 6 bases were exchanged for an SpeI cleavage site (italicized) in the forward primer. Secondly, to clone the hemA[KK] mutant, a further 6 bases were replaced by a DNA sequence which is 6 bases in length and which codes for two lysines (underlined). The insertion site is chosen in such a way that the “KK insertion” comes to be at the third and fourth positions of the N terminus of the amino acid sequence. Since the genetic code is degenerate, the codon usage of B. megaterium was used for determining the most likely sequence. The codon usage states the probability with which a certain base triplet codes for an amino acid in the genome of the organism. In the case of B. megaterium, “AAA”, with a percentage usage of 76%, is the most frequent triplet for lysine. A KpnI cleavage site was introduced into the reverse primer. The primers were synthesized by MWG, Ebersbach.

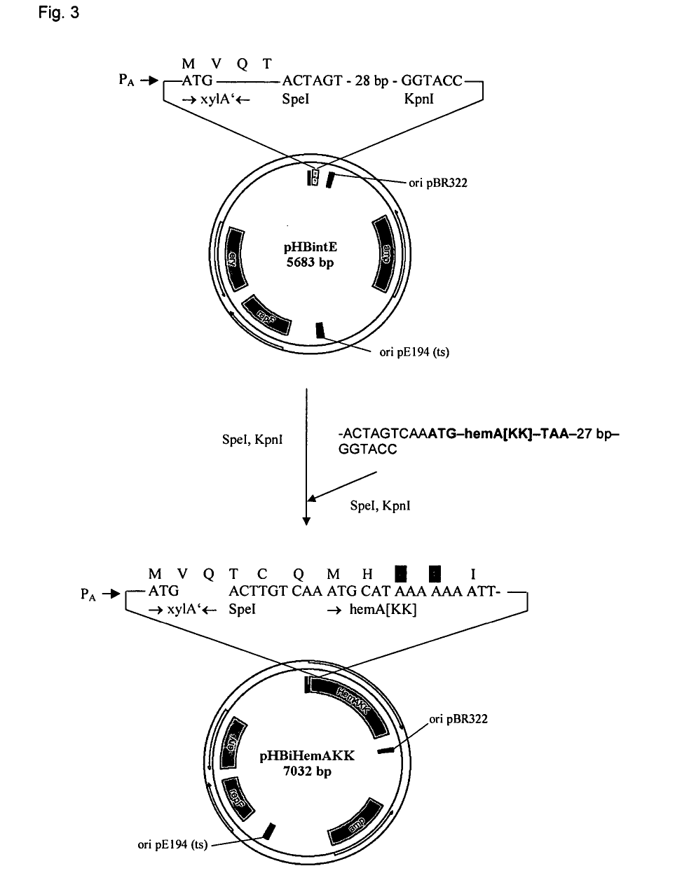

The hemA[KK] mutant which had been amplified via PCR was purified using a PCR Purification Kit from Quiagen, cut with SpeI and KpnI and then again purified. The plasmid pHBintE was likewise cut with SpeI and KpnI and purified using a PCR Purification Kit from Quiagen. After the concentration has been determined, these two fragments were ligated in a vector/insert ratio of 1:4 to give the integration vector with the name pHBiHemAKK. The cloning strategy for the plasmid pHBiHemAKK is shown schematically in FIG. 3.

Since pHBiHemAKK differs from pHBintE essentially only by the insertion of the hemA[KK] mutant, it retains the properties of pHBintE. Furthermore present are the ampicillin resistance and the erythromycin resistance for selection in E. coli and in B. megaterium, respectively. The origin pBR322 serves for the replication in E. coli, and the temperature-sensitive origin pE194ts and repF for replication in B. megaterium. Xyl A′ and the hemA[KK] mutant were ligated to give a translational fusion and are under the control of the xyl promoter. As the result of the hemA[KK] insertion, pHBiHemAKK has a segment which is homologous to the B. megaterium chromosome, and thus additionally the possibility of integrating into the chromosome via single crossing-over recombination.

The temperature-sensitive origin pE194ts is of importance for the selection of this integration event. Since the plasmid is replicated at 30° C., B. megaterium transformants can be selected for erythromycin at this temperature. When the temperature is increased to 42° C., the plasmid can no longer be replicated. This means that only those transformants which have integrated the plasmid, and thus the erythromycin resistance, into their chromosome are capable of growth.

The integration of pHBiHemA[KK] into the B. megaterium chromosome thus makes possible a xylose-inducible overexpression of all of the hemAXCDBL operon. Moreover, as the result of the feedback-deregulated mutant of the HemA protein, the plasmid comprises an improved possibility of overproducing vitamin B₁₂. The B. megaterium strain DSMZ509 with the integrated plasmid pHBiHemA[KK] is hereinbelow referred to as HBBml.

Construction of pHBasHemZ

Starting from genomic DNA isolated from B. megaterium DSMZ509, PCR and the primers stated hereinbelow were used to amplify, by customary laboratory practice, a 129 bp BamHI/SpeI fragment from the 5′ region of the mRNA of the hemZ gene in the form of an antisense RNA.

Primer forward (ashemZ): comprises BamHi cleavage
site
5′-GCGGGATCCCTTGAACTGAGCACCTTGACCGG-3′
Primer reverse (ashemZ): comprises SpeI cleavage
site
5′-TCGACTAGTCGGACGTAAAAAACGTTCATCTTCTATACC-3′

PCR Conditions:

7 min/95° C.
30 times:
1 min/95° C.
1 min/64° C.
1 min/72° C.
7 min/72° C.

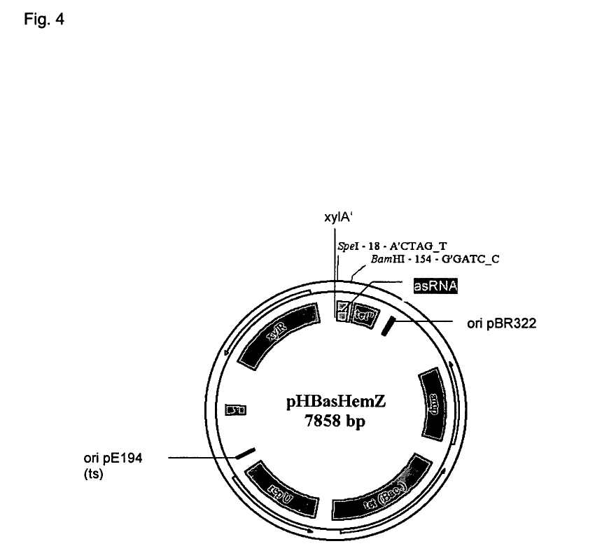

Thereafter, the amplified BamHI/SpeI antisense RNA fragment was purified and cloned into the vector pWH1520 (Rygus, T. et al., 1991, Appl. Microbiol. Biotechnol., 35: 594-599) which had previously been linearized with the restriction endonucleases SpeI and BamHI. The resulting plasmid pHBasHemZ, which comprises an antisense hemZ RNA under the control of the xylA promoter, is shown in FIG. 4.

The inserted antisense hemZ RNA as shown in SEQ ID No. 3 has a length of 129 bp and starts 82 nucleotides before the start codon of the actual hemZ gene. An overview over the position of the antisense hemZ RNA can be found hereinbelow:

```
                                                      →asRNA (129 bp) −35
5′CGTTTGTTTCCTGTCCGCGCATTCCCTTGAACTGAGCACCTTGACCGGACATA
                                −10 RBS
CGTAGGTTTTGTAAACTGATTACTTAGATAGAATTGATTTGAAAGGTGATTATA
Start hemZ [symbol]
TTGAACATTTATATAAAAGGTATAGAAGATGAACGTTTTTTACGTCCGCTTCAC
CGAATTTCAGATTTGTTTTTTGAAGAAAGCAACGTC-3′
```

Accordingly, the transcript of the antisense RNA together with the hemZ mRNA forms a double-stranded RNA and thus blocks the ribosomal binding site of the hemz gene for the ribosomes.

Transformation and Integration of pHBiHemAKK into Bacillus megaterium

In order to be able to integrate the present integration vector into the chromosome, B. megaterium DSMZ509 was first transformed with pHBiHemAKK by means of protoplast transformation. The transformed strain was grown on agar plates with addition of erythromycin (1 μg/ml and 75 μg/ml). The culture temperature chosen was 30° C. since the plasmid is capable of replication at this temperature.

After 24 hours' growth, colonies have been identified for all stated erythromycin concentrations. Also, the transfer of some colonies onto agar plates with added erythromycin (75 μg/ml) resulted in growth after 24 hours, with the abovementioned conditions prevailing. The transformation of pHBiHemAKK into B. megaterium DSMZ509 was thus successful.

A 110 ml LB culture supplemented with 5 μg/ml erythromycin and 0.23% xylose was inoculated with these transformants and incubated at 30° C. under aerobic conditions (shaking at 250 rpm). After approx. 12 hours, the temperature was raised to 42° C. so that further replication of the plasmid was no longer possible and pressure to integrate was built up. After a further 12 h, the culture was transferred into in each case fresh LB medium for a total of 3 days and incubation was continued at 42° C. After this time, good colonization of the LB medium was observed; this indicated the integration of the plasmid into the chromosome since transformants with freely replicable plasmids would not have been possible to pass on the plasmid by replication under these conditions.

Growth Behavior of the Genetically Modified B. megaterium DSMZ509

B. megaterium DSMZ509 with intergrated pHBiHemA[KK] under aerobic conditions

To check the growth capability of the new strain, growth curves were recorded in LB medium with addition of 5 μg/ml erythromycin and 0.23% xylose at 42° C. under aerobic conditions. Good aerobic growth for the pHBiHemAKK integration transformant is shown when a small amount of xylose is present in the medium.

B. megaterium DSMZ509 with pHBasHemZ in Shift Experiment

In what are known as “shift experiments”, B. megaterium is initially grown under aerobic conditions in order to achieve a high cell density. Thereafter, the culture is, at the end of the exponential phase, transferred into anaerobic conditions since the bacteria achieve a substantially higher vitamin B₁₂ content under anaerobic conditions (Barg, H., 2000, Vitamin B12-Produktion durch Bacillus megaterium [Vitamin B12 production by Bacillus megaterium], diploma thesis, Albert Ludwig University, Freiburg).

The untransformed strain B. megaterium DSMZ509 and the transformants DSMZ509-pWH1520 and DSMZ509-pHBasHemZ were compared when grown with Mopso minimal medium with glucose as the carbon source. Again, 30 μg/ml tetracyclin were added to the transformant cultures. Induction with 0.5% (w/v) xylose was effected after 9 hours' growth; this means 1 hour prior to the shift from aerobic to anaerobic conditions.

Since no pronounced difference in the growth of the antisense hemZ RNA forming transformant and the comparative transformant was found, growth comparisons with addition of CoCl₂ and ALA were carried out. Addition of ALA should also result in an increased metabolite flux into the hem synthetic pathway. Thus, inhibition by the antisense hemZ RNA should reveal a pronounced difference in growth with regard to the comparative transformant. In this shift experiment, the cultures of the transformants again received an addition of tetracyclin of 30 μg/ml and additionally an addition of 250 μM CoCl₂ and 298 μM ALA. Induction with 0.5% (w/v) xylose was effected after 10 hours' growth (corresponds to 1 hour before the shift).

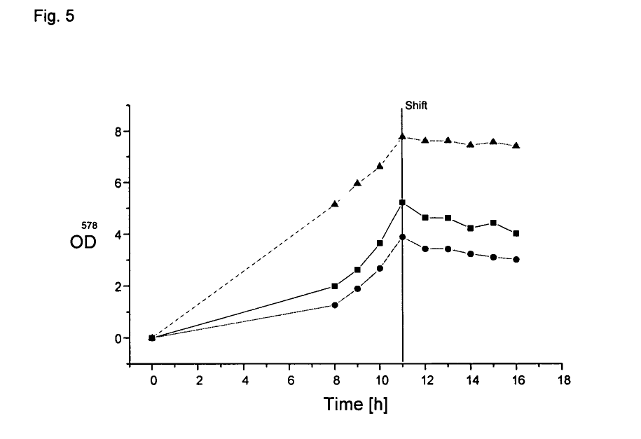

FIG. 5 shows that, with addition of 298 μM ALA and 250 μM CoCl₂, the growth of B. megaterium DSMZ509-pHBasHemZ (-!-) over its entire course is markedly worse than in the case of the comparative transformant DSMZ509-pWH1520 (-+-). This means that a reduction of the hem formation by the antisense RNA has taken place. The addition of ALA (the precursor molecule of all tetrapyrroles) appears to stimulate tetrapyrrole synthesis to such an extent that the inhibition of coproporphyrinogen-Ill oxidase (HemZ) by the antisense hemZ RNA affects growth.

The cell densities achieved with addition of ALA and CoCl₂ are lower than in the case of growth without these additions. In the case of the transformants, the cell densities achieved had an OD₅₇₈ of 3.9 for the antisense transformant and of 5.2 for the comparative transformant (without additions: 8.7 and 8.8). The untransformed strain DSMZ509 (-▴-) shows the best growth over its entire course and, at the point in time of shift, had the highest cell density with an OD₅₇₈ of 7.8 (without additions: 10.8).

The growth disadvantage of the transformants in comparison with the untransformed strain is caused by the additional replication of the plasmids and by the addition of antibiotics to the medium.

Bacillus megaterium DSMZ509-DHBasHemZ with Addition of Cobalt and ALA Under Aerobic Conditions

In the above shift experiments, only one hour's post-induction growth took place under aerobic conditions. Since hem is required mainly under aerobic conditions, a growth comparison of the two B. megaterium transformants DSMZ509-pWH1520 and DSMZ509-pHBasHemZ was carried out in Mopso minimal medium with glucose as the carbon source and with the addition of 250 μM CoCl₂ and 298 μM ALA under aerobic conditions. Induction with 0.5% xylose (w/v) was carried out at an OD₅₇₈ of 2.

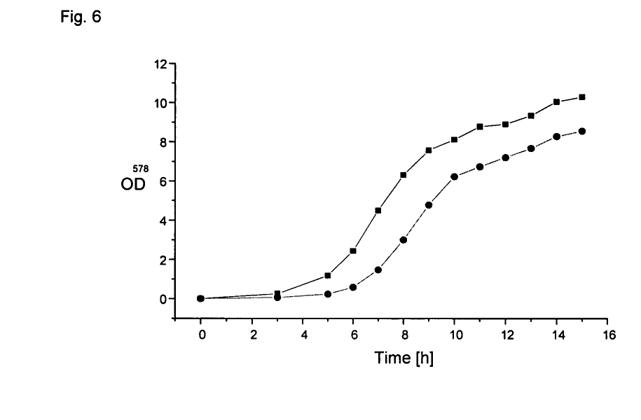

FIG. 6 confirms that the antisense hemZ RNA formed is growth-inhibitory. Again, the growth of DSMZ509-pHBasHemZ (-!-) is poorer at each point in time than that of the comparative transformant (-+-). Thus, the antisense-hemZ-RNA-forming transformant reaches a maximum OD₅₇₈ of 8.3, while the maximum OD₅₇₈ of the comparative transformant is 10.0. This means that an inhibition of coproporphyrinogen-Ill oxidase has taken place.

Quantitative Vitamin B₁₂ Analysis

Two different methods were employed for the quantitative determination of vitamin B₁₂. Firstly, the determination was based on the growth of S. typhimurium metE cysG dual mutants, and, secondly, using the RIDASCREEN®FAST vitamin B₁₂ ELISA assay from r-biopharm in conjunction with the Fusion Plate Reader from Packard.

Vitamin B12 Determination Using S. typhimurium metE cvsG Dual Mutants

Samples were taken from B. megaterium cultures during different growth phases. After the determination of the OD₅₇₈, the cells were separated from the medium by means of centrifugation (4000×g; 15 min; 4° C.). The cell sediments obtained and the media removed were subsequently freeze-dried. S. typhimurium metE cysG dual mutants were incubated at 37° C. overnight on methionin- and cystein-comprising minimal medium, scraped away from the plate and washed using 40 ml of isotonic NaCl solution. After centrifugation, the cell sediment was resuspended in isotonic saline. The washed bacterial culture was mixed carefully with 400 ml of cystein-comprising minimal medium agar with a temperature of 47-48° C.

10 μl of the B. megaterium samples which had been resuspended in deionized sterile water and boiled for 15 minutes in a water bath were placed on the cooled plates and incubated for 18 hours at 37° C. The diameters of the salmonella colonies which had grown are now proportional to the vitamin B12 content of the B. megaterium samples applied. A comparison with a calibration curve, established with the addition of 0.01, 0.1, 1, 10 and 40 pmol vitamin B12, allowed a conclusion regarding the vitamin B12 content in the test samples. Using this standard method, small amounts of vitamin B12 in biological materials can be detected rapidly and with a high degree of reproducibility.

Vitamin B12 determination with the ELISA assay Principle of the assay: The assay is based on an antigen/antibody reaction, where the wells of a microtiter plate are coated with specific antibodies against vitamin B12. After addition of enzyme-labeled vitamin B12 (enzyme conjugate) and sample solutions, or vitamin B12 standard solutions, free and enzyme-labeled vitamin B12 compete for the vitamin B12 antibody binding sites (competitive enzyme immunoassay). Unbound enzyme-labeled vitamin B12 is subsequently removed in a wash step. Detection is by addition of substrate/chromogen solution (tetramethylbenzidin/urea peroxide). Bound enzyme conjugate converts the chromogen into a blue end product. Addition of the stop reagent leads to a color change from blue to yellow. This is measured photometrically at 450 nm. Thus, the absorbance of the solution is inversely proportional to the vitamin B12 concentration in the sample. Procedure: 2 ml samples were removed at different times of growth from the B. megaterium liquid culture to be measured, and the OD578 was determined. The cells were separated from the medium by subsequent centrifugation (4000×g; 5 min; 4° C.), the supernatant was discarded and the sediment was freeze-dried. The reagents required (standards, enzyme conjugate and wash buffer concentrate), of the test kit, were then brought to room temperature and prepared and diluted as described in the accompanying protocol. The samples were prepared immediately before carrying out the test. To this end, the freeze-dried cells were resuspended in 0.5 ml of sterile deionized water. Quantitative cell disrupture was achieved by addition of 50 μl of lysosyme solution (1 mg/ml), followed by incubation (30 min; 37° C.; shaking at 300 rpm), sonication (5 min) and boiling (3 min; 100° C.). The samples were then ice-cooled to room temperature and centrifuged (4000×g; 5 min; 15° C.). The supernatant was removed and diluted 1:5 with the sample dilution buffer. Then, in each case 50 μl of the dilute samples and of the dilute vitamin B12 standards were pipetted into the cavities of the microtiter plate. After addition of 50 μl of the dilute enzyme conjugate, the samples were mixed (shaker function in the fusion device) and incubated (15 min; RT). After the incubation, the cavities were emptied by tapping the microtiter plate and washed using 250 μl of wash buffer per cavity. Again, the cavities were emptied by tapping and the wash step was repeated twice. This was followed by the equitemporal addition of two drops of stop reagent per cavity, mixing and incubation in the dark for 10 minutes at room temperature. Following the equitemporal addition of two drops of the stop reagent per cavity, the absorbance at 450 nm was measured in the fusion device from Packard.
For the evaluation, the percentage absorbance was calculated as follows:

$$\frac{Absorbance of standard or sample}{Absorbance of blank standard} \cdot 100 = absorbance in \%$$

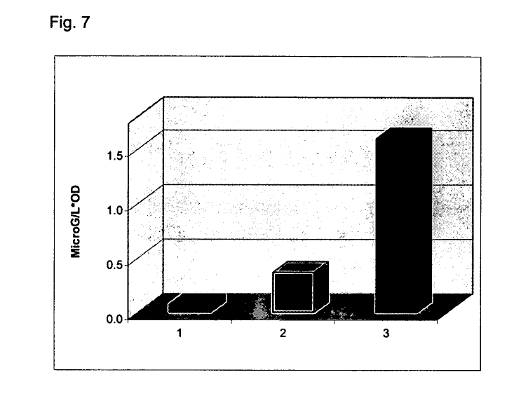

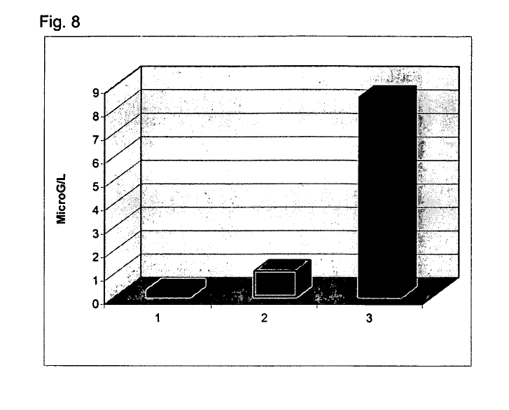

A calibration line was then established by plotting the absorbance in % versus log (ppb). Thereafter, it was possible to indicate the vitamin B12 content of the samples in μg/(I×OD) via the linear equation, the dilution factor and the known cell density (OD578). ELISA vitamin B12 determination of Bacillus megaterium DSMZ509 with integrated pHBiHemAKKTo check the vitamin B12 contents of cultures with xylose-inducible hemA[KK]XCDBL operon, the integrated strain, and, as comparative strains, DSMZ509 and Z509-pWH1520-cobA, were grown aerobically. After ten hours' growth and 5 hours' post-induction (t=5), the samples were digested in accordance with the established ELISA vitamin B12 assay, and the vitamin B12 contents were measured. Owing to their reddish-brown coloration in comparison with the yellowish DSMZ509 comparative culture, the centrifuged cell pellets already suggested an increased tetrapyrrole content. As shown in FIGS. 7 and 8, this is confirmed by the ELISA assay. The suspected overexpression of the hemA[KK]XCDBL operon led to an increase in the vitamin B12 content of from 0.07 (g/l*OD578 of the wild strain (DSMZ509) to 1.59 (g/l*OD578 in the integrated strain. This corresponds to an increase by the factor 22. If the increase is calculated in (g/l, the result is no less than an increase by the factor 30 (from 0.26 (g/l in the case of DSMZ509 to 8.51 μg/l in the case of DSMZ509 with integrated pHBiHemAKK). ELISA vitamin B₁₂ determination of Bacillus megaterium DSMZ509-pHBasHemZ In the shift experiments, samples of the B. megaterium transformants DSMZ509-pWH1520 and DSMZ509-pHBasHemZ were taken three hours (T=3) and six hours (T=6) after the induction with xylose. These samples were analyzed for vitamin B₁₂ with the aid of an ELISA assay from R-Biopharm, which is described in detail in the section materials and methods.

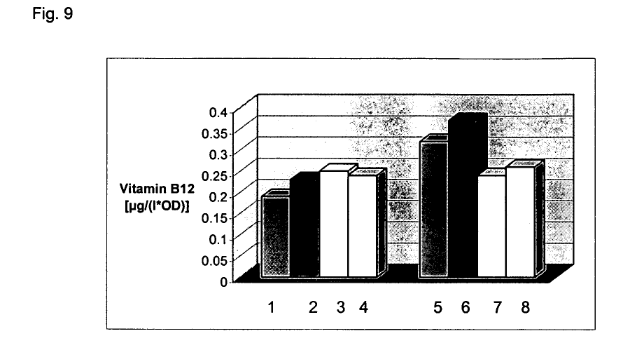

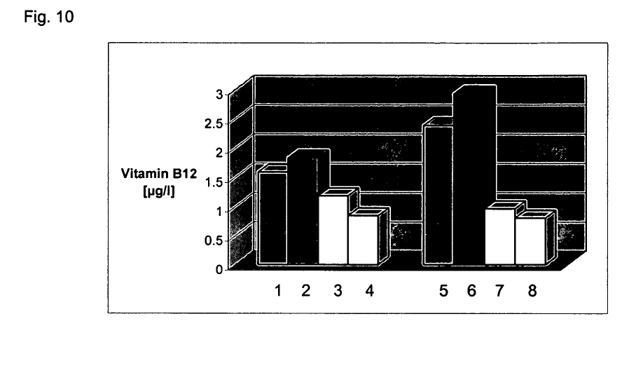

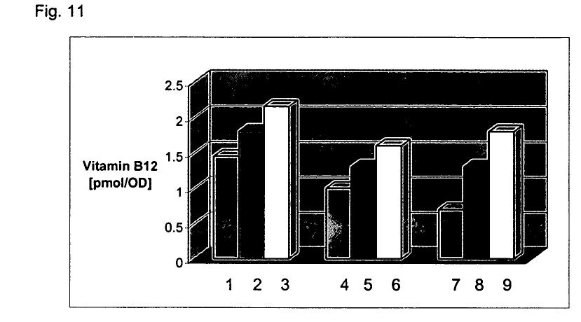

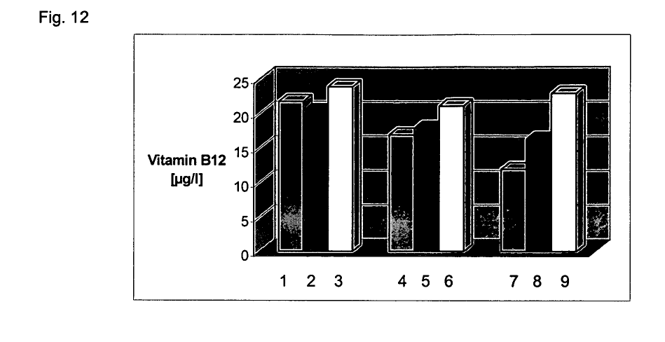

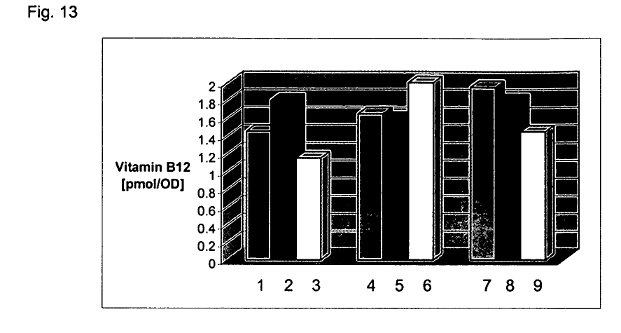

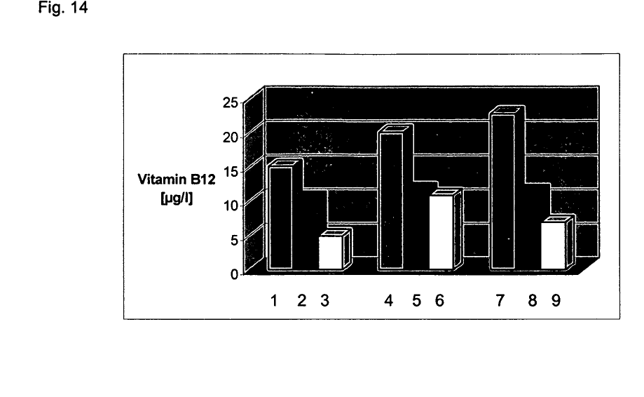

In the shift experiments, the transfer from aerobic to anaerobic conditions took place one hour post-induction. FIG. 9 and FIG. 10 show the results of the vitamin B₁₂ determination for growth with glucose (1, 2, 5, 6) and for growth with glucose with addition of 298 μM ALA and 250 μM CoCl₂ (3, 4, 7, 8). FIG. 9 shows the vitamin B₁₂ concentrations based on the cell density of the culture in question. It can be seen that, in three out of four cases (Nos 2, 6 and 8), DSMZ509-pHBasHemZ has formed more vitamin B₁₂ than the comparative transformant (Nos 1, 5 and 7). Thus, the vitamin B₁₂ content of the antisense-hemZ-RNA-forming transformant in the case of growth without additions at the time T=3 (No. 2) is 21% higher, and in the case of T=6 (No. 6) 16% higher than in the case of the comparative transformant. In the case of growth with cobalt and ALA, DSMZ509-pHBasHemZ forms 10% more vitamin B₁₂ than DSMZ509-pWH1520 at six hours post-induction (No. 8). In FIG. 10, the difference in the vitamin B₁₂ concentrations between the cultures without addition of cobalt and ALA and those with addition is more pronounced. The reasons are the higher cell densities which were achieved with growth without additions. Bioassay vitamin B₁₂ determination of Bacillus megaterium DSMZ509-PHBasHemZ To determine the vitamin B₁₂ contents, in the shift experiments, by means of bioassay, samples were taken at three different points in time. Sampling took place 1.) at the point in time of induction (T=0), 2.) three hours post-induction (T=3) and 3.) six hours post-induction during the stationary phase (T=stationary). Here, the shift from aerobic to anaerobic conditions took place one hour post-induction. The vitamin B₁₂ contents of the B. megaterium strains DSMZ509, DSMZ509-pWH1520 and DSMZ509-pHBasHemZ were measured. Firstly for growth with glucose, secondly for growth with glucose and with the addition of 250 μM CoCl₂ and 298 μM ALA. The determination was carried out using the S. typhimurium metE cysG dual mutant AR3612. The vitamin B₁₂ content is shown in pmol/OD₅₇₈ and in μg/l (FIGS. 11-12). FIG. 11 shows that, in the case of growth with glucose, the vitamin B₁₂ contents based on the cell density are highest at any point in time for DSMZ509-pHBasHemZ (Nos 3, 6 and 9). Again, this shows that the inhibition of hem synthesis results in an increased metabolite flux toward the synthesis of vitamin B₁₂. The diagram showing the results in μg per liter of bacterial culture (FIG. 12) shows that, again, the antisense-hemZ-RNA-transcribing transformant (Nos 3, 6 and 9) has produced the overall highest amounts of vitamin B₁₂, although a low cell density was obtained with this transformant. FIG. 13 shows that, with addition of CoCl₂ and ALA to the medium, the antisense-hemZ-RNA-transcribing transformant achieves the highest vitamin B₁₂ content after as little as three hours' post-induction (No. 6). In a first attempt to explain this phenomenon, it appears that the induction does not immediately lead to maximum plasmid replication, but needs a start-up phase. On considering the vitamin B₁₂ contents per liter of bacterial culture, it can be seen that, as the result of its better growth, the untransformed strain DSMZ509 produces more vitamin B₁₂ than the other two, transformed, strains (FIG. 14). Relative coproporphyrinogen III determination: methods Fluorescence spectra 2 ml samples were removed at different times of growth from the B. megaterium liquid culture to be measured, and the OD₅₇₈ was determined. The cells were separated from the medium by subsequent centrifugation (4000×g; 5 min; 4° C.), the supernatant was discarded, and the sediment was freeze-dried. Immediately before a measurement, the samples were resuspended in 1 ml of sterile deionized water, and the optical densities were subsequently adjusted by dilution with water. 1 ml of these adjusted samples were then treated with 50 μl of lysosyme (1 mg/ml) and incubated in the shaker for 30 minutes at 37° C. and 300 rpm. Then, the samples were placed for 10 minutes into an ultrasonic bath and thereafter centrifuged for 3 minutes at 4000×g. The fluorescence measurement was performed on the supernatant, with the following settings: Start: 430 nm End: 680 nm Excitation: 409 nm Ex Slit 12 nm Em Slit: 12 nm Scan Speed: 200 nm/min The growth curves with addition of CoCl₂ and ALA gave the first indications that the synthesis of hem is inhibited by antisense hemZ RNA. The xylose-inducible antisense hemZ RNA inhibits the ribosomal binding site by occupying the hemZ mRNA and thus prevents translation into hemZ. This leads to reduced formation of the hemZ protein, which catalyzes the reaction from coproporphyrinogen III to protoporphyrinogen IX. Since the actual metabolite flux is interrupted at this point, coproporphyrinogen III accumulates. The direct detection of coproporphyrinogen III in samples proves difficult since coproporphyrinogen III is oxidized in the air to give coproporphyrin III. Preliminary experiments revealed that the fluorescence spectrum of coproporphyrin III has emission peaks of approximately 579 nm and approximately 620 nm. Accordingly, it should be possible to detect relative amounts of coproporphyrinogen III indirectly with the aid of fluorescence spectra, measuring the oxidized form (coproporphyrin III).

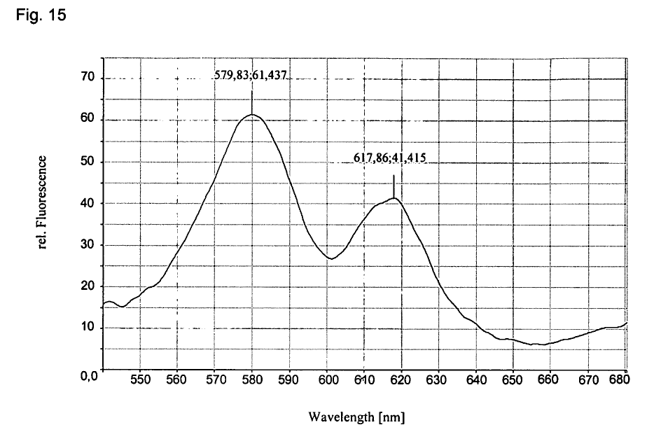

To demonstrate the different relative amounts of coproporphyrinogen III in DSMZ509-pHBasHemZ and in the comparative transformant DSMZ509-pWH1520, fluorescence measurements were carried out. Samples of the transformants DSMZ509-pHBasHemZ and DSMZ509-pWH1520 were taken three hours post-induction with 0.5% (w/v) xylose from the growth experiment with Mopso minimal medium with glucose as the carbon source and an addition of 298 μM ALA and 250 μM CoCl₂. First, the optical densities of the samples were adjusted by dilution with water. Thereafter, the cells were disrupted and the cell extract was measured. The individual spectra of these samples showed a similar course, the spectrum of the transformant harboring the antisense hemZ RNA always showing higher fluorescence levels. The difference of the two spectra appears to become wider at the peaks (at 579 nm and 612 nm). To demonstrate this difference, FIG. 15 shows the differential spectrum of the two samples (DSMZ509-pHBasHemZ minus DSMZ509-pWH1520). The peaks at 579.83 nm and 617.86 nm show that the antisense-RNA-forming transformant accumulates coproporphyrinogen III in comparison with the comparative transformant. This is an unambiguous proof of the fact that an inhibition of hem biosynthesis has been achieved with the aid of antisense RNA. Using DSMZ509-pHBasHemZ, it is thus possible to prevent the hem biosynthetic pathway by targeted induction with xylose, which, in turn, should permit an uninterrupted metabolite flux toward the vitamin B₁₂ synthetic pathway.

Key to the Figures

The present invention is explained in greater detail by means of the figures below.

FIG. 1 shows the schematic representation of the cloning of the integrative plasmid pHBintE for B. megaterium. The starting plasmids pWH1967E and pMM1520 were cut with the endonucleases PstI and HindIII. The 4198 bp fragment (between PstI-2786 and HindIII-6984) of pWH1967E and 1485 bp fragment (between HindIII-7212 and PstI-1307) of pMM1520 were eluted and ligated.

FIG. 2 shows a representation of the first 27 amino acids of the alignment report for 1.) S. typhimurium HemA, 2.) B. megaterium HemA and 3.) B. megaterium HemAKK. Underlined: Insertion of two positively charged lysine residues (KK) at positions 3 and 4 of the N terminus.

FIG. 3 shows a schematic representation of the cloning strategy of the plasmid pHBiHemAKK. The PCR-amplified hemA[KK] mutant and the vector pHBintE were each cut with SpeI and KpnI, and the resulting cohesive ends were ligated to give the integration vector pHBiHemAKK.

FIG. 4 shows a schematic representation of the plasmid pHBasHemZ. The cleavage sites SpeI and BamHI, which are shown in the representation, were used for inserting the antisense RNA.

FIG. 5 shows the growth behavior of the B. megaterium-strain DSMZ509 and of transformants of this strain at 37° C. in Mopso minimal medium with glucose as the carbon source and addition of 298 μM ALA and 250 μM CoCl₂. Shift from aerobic to anaerobic growth took place at the end of the exponential phase (after 11 h). Growth of DSMZ509 untransformed (-▴-), DSMZ509 pWH1520 (-+-) and DSMZ509 pHBasHemZ (-!-). Induction of the gene expression of the xylA promoter on pHBasHemZ and pWH1520 took place by addition of 0.5% (w/v) xylose after 10 hours' growth. At the stated times, samples were taken, and the optical density at 578 nm was determined.

FIG. 6 shows the growth behavior of the B. megaterium strain DSMZ509-pWH1520 (-+-) and DSMZ509-pHBasHemZ (-!-) in Mopso minimal medium with glucose as the carbon source and addition of 298 μM ALA and 250 μM CoCl₂ under aerobic growth conditions. Induction was carried out by addition of 0.5% (w/v) xylose at an OD₅₇₈ of 2. At the stated times, samples were taken, and the optical density at 578 nm was determined.

FIG. 7 shows the vitamin B₁₂ content in μg/l*OD under aerobic growth conditions of B. megaterium DSMZ509 (1), DSMZ509-pWH1520-cobA (2) and DSMZ509 with integrated pHBiHemAKK (3) in LB medium, measured in an ELISA assay. Induction was carried out with 0.5% (w/v) xylose after 5 hours' growth; cells were harvested after 10 hours' growth.

1=DSMZ509

2=DSMZ509-pWH1520-cobA

3=DSMZ509 with integrated pHBiHemAKK

FIG. 8 shows the vitamin B₁₂ content in μg/l under aerobic growth conditions of B. megaterium DSMZ509 (1), DSMZ509-pWH1520-cobA (2) and DSMZ509 with integrated pHBiHemAKK (3) in LB medium, measured in an ELISA assay. Induction was carried out with 0.5% (w/v) xylose after 5 hours' growth; cells were harvested after 10 hours' growth.

1=DSMZ509

2=DSMZ509-pWH1520-cobA

3=DSMZ509 with integrated pHBiHemAKK

FIG. 9 the vitamin B₁₂ production in the shift experiment of B. megaterium DSMZ509-pWH1520 and DSMZ509-pHBasHemZ in Mopso minimal medium with glucose as the carbon source measured in an ELISA test. Induction was carried out with 0.5% (w/v) xylose after 9 hours' and 10 hours' growth (1, 2, 5, 6 and 3, 4, 7, 8 respectively). The shift from the aerobic to the anaerobic took place one hour post-induction. The vitamin B₁₂ content is shown in μg per liter of bacterial culture and OD₅₇₈.

1=DSMZ509-pWH1520 without additions, 3 h post-induction.

2=DSMZ509-pHBasHemZ without additions, 3 h post-induction.

3=DSMZ509-pWH1520 with addition of 250 μM CoCl₂ and 298 μM ALA, 3 h post-induction.

4=DSMZ509-pHBasHemZ with addition of 250 μM CoCl₂ and 298 μM ALA, 3 h post-induction.

5=DSMZ509-pWH1520 without additions, 6 h post-induction.

6=DSMZ509-pHBasHemZ without additions, 6 h post-induction.

7=DSMZ509-pWH1520 with addition of 250 μM CoCl₂ and 298 μM ALA, 6 h post-induction.

8=DSMZ509-pHBasHemZ with addition of 250 μM CoCl₂ and 298 μM ALA, 6 h post-induction.

FIG. 10 shows the vitamin B₁₂ production in the shift experiment of B. megaterium DSMZ509-pWH1520 and DSMZ509-pHBasHemZ in Mopso minimal medium with glucose as the carbon source measured in an ELISA assay. Induction was carried out with 0.5% (w/v) xylose after 9 hours' and 10 hours' growth (1, 2, 5, 6 and 3, 4, 7, 8 respectively). The shift from aerobic to anaerobic took place one hour post-induction. The vitamin B₁₂ content is shown in μg per liter of bacterial culture.

1=DSMZ509-pWH1520 without additions, 3 h post-induction.

2=DSMZ509-pHBasHemZ without additions, 3 h post-induction.

3=DSMZ509-pWH1520 with addition of 250 μM CoCl₂ and 298 μM ALA, 3 h post-induction.

4=DSMZ509-pHBasHemZ with addition of 250 μM CoCl₂ and 298 μM ALA, 3 h post-induction.

5=DSMZ509-pWH1520 without additions, 6 h post-induction.

6=DSMZ509-pHBasHemZ without additions, 6 h post-induction.

7=DSMZ509-pWH1520 with addition of 250 μM CoCl₂ and 298 μM ALA, 6 h post-induction.

8=DSMZ509-pHBasHemZ with addition of 250 μM CoCl₂ and 298 μM ALA, 6 h post-induction.

FIG. 11 shows the vitamin B₁₂ production in the shift experiment of B. megaterium DSMZ509, DSMZ509-pWH1520 and DSMZ509-pHBasHemZ in Mopso minimal medium with glucose as the carbon source. The shift from the aerobic to the anaerobic took place one hour post-induction. Induction was performed with 0.5% (w/v) xylose after 9 hours' growth. The vitamin B₁₂ content per cell biomass is shown in pmol/OD₅₇₈.

1=DSMZ509, at the time of induction.

2=DSMZ509-pWH1520, at the time of induction.

3=DSMZ509-pHBasHemZ, at the time of induction.

4=DSMZ509, 3 h post-induction.

5=DSMZ509-pWH1520, 3 h post-induction.

6=DSMZ509-pHBasHemZ, 3 h post-induction.

7=DSMZ509, 6 h post-induction.

8=DSMZ509-pWH1520, 6 h post-induction.

9=DSMZ509-pHBasHemZ, 6 h post-induction.

FIG. 12 shows the vitamin B₁₂ production in the shift experiment of B. megaterium DSMZ509, DSMZ509-pWH1520 and DSMZ509-pHBasHemZ in Mopso minimal medium with glucose as the carbon source. The shift from the aerobic to the anaerobic took place one hour post-induction. Induction was performed with 0.5% (w/v) xylose after 9 hours' growth. The vitamin B₁₂ content is shown in μg per liter of bacterial culture.

1=DSMZ509, at the time of induction.

2=DSMZ509-pWH1520, at the time of induction.

3=DSMZ509-pHBasHemZ, at the time of induction.

4=DSMZ509, 3 h post-induction.

5=DSMZ509-pWH1520, 3 h post-induction.

6=DSMZ509-pHBasHemZ, 3 h post-induction.

7=DSMZ509, 6 h post-induction.

8=DSMZ509-pWH1520, 6 h post-induction.

9=DSMZ509-pHBasHemZ, 6 h post-induction.

FIG. 13 shows the vitamin B₁₂ production in the shift experiment of B. megaterium DSMZ509, DSMZ509-pWH1520 and DSMZ509-pHBasHemZ in Mopso minimal medium with glucose as the carbon source, with addition of 298 μM ALA and 250 μM CoCl₂. The shift from the aerobic to the anaerobic took place one hour post-induction. Induction was performed with 0.5% (w/v) xylose after 10 hours' growth. The vitamin B₁₂ content per cell biomass is shown in pmol/OD₅₇₈.

1=DSMZ509, at the time of induction.

2=DSMZ509-pWH1520, at the time of induction.

3=DSMZ509-pHBasHemZ, at the time of induction.

4=DSMZ509, 3 h post-induction.

5=DSMZ509-pWH1520, 3 h post-induction.

6=DSMZ509-pHBasHemZ, 3 h post-induction.

7=DSMZ509, 6 h post-induction.

8=DSMZ509-pWH1520, 6 h post-induction.

9=DSMZ509-pHBasHemZ, 6 h post-induction

FIG. 14 shows the vitamin B₁₂ production in the shift experiment of B. megaterium DSMZ509, DSMZ509-pWH1520 and DSMZ509-pHBasHemZ in Mopso minimal medium with glucose as the carbon source, with addition of 298 μM ALA and 250 μM CoCl₂. The shift from the aerobic to the anaerobic took place one hour post-induction. Induction was performed with 0.5% (w/v) xylose after 9 hours' growth. The vitamin B₁₂ content is shown in μg per liter of bacterial culture.

1=DSMZ509, at the time of induction.

2=DSMZ509-pWH1520, at the time of induction.

3=DSMZ509-pHBasHemZ, at the time of induction.

4=DSMZ509, 3 h post-induction.

5=DSMZ509-pWH1520, 3 h post-induction.

6=DSMZ509-pHBasHemZ, 3 h post-induction.

7=DSMZ509, 6 h post-induction.

8=DSMZ509-pWH1520, 6 h post-induction.

9=DSMZ509-pHBasHemZ, 6 h post-induction

FIG. 15 shows the differential fluorescence spectrum of B. megaterium DSMZ509-pHBasHemZ minus the fluorescence spectrum of DSMZ509-pWH1520 with excitation at 409 nm. The emission peaks at 579 nm and 618 nm indicate coproporphyrin III, and thus an accumulation of the metabolite in DSMZ509-pHBasHemZ in comparison with DSMZ509-pWH1520.

1. A genetically modified Bacillus megaterium strain comprising a gene hemA[KK] as shown in SEQ ID No. 4 coding for a feedback-resistant glutamyl-tRNA reductase and/or part of the nucleotide sequence as shown in SEQ ID No. 1 (hemZ) as antisense RNA (ashemZ).

2. The genetically modified Bacillus megaterium strain according to claim 1, comprising a gene hemA[KK] as shown in SEQ ID No. 4, organized in a hemA[KK]XCDBL operon, and/or an antisense RNA (ashemZ) as shown in SEQ ID No. 3.

3. The genetically modified Bacillus megaterium strain according to claim 1, in which the hemA[KK] gene is integrated into the chromosome of the bacterium.

4. The genetically modified Bacillus megaterium strain according to claim 1, in which the part of the hemZ gene is present as plasmid-encoded antisense RNA (ashemZ) in an increased copy number.

5. The genetically modified Bacillus megaterium strain according to claim 1, where the hemA[KK] gene, organized in the hemA[KK]XCDBL operon and/or the part of the hemZ gene as antisense RNA (ashemZ) is under the control of an inducible promoter.

6. The genetically modified Bacillus megaterium strain according to claim 5, which comprises the xylA promoter as inducible promoter.

7. An integrative vector comprising a gene hemA[KK] coding for a feedback-resistant glutamyl-tRNA reductase as shown in SEQ ID No. 4 and, in operable linkage therewith, sequences for the induced gene expression, selection, replication and/or integration into the chromosome of the host cell.

8. The integrative vector according to claim 7, characterized in that it comprises a genetically modified nucleotide sequence of the hemA gene (hemA[KK]), which nucleotide sequence codes for a feedback-resistant glutamyl-tRNA synthase whose amino acid sequence comprises an insertion of at least two positively charged amino acids.

9. The integrative vector according to claim 8, characterized in that it comprises a genetically modified nucleotide sequence of the hemA gene (hemA[KK]), which nucleotide sequence codes for a feedback-resistant glutamyl-tRNA synthase whose amino acid sequence comprises, at positions 3 and 4 of the N terminus, an insertion of two positively charged amino acids.

10. The integrative vector according to claim 8, characterized in that the inserted positively charged amino acids are lysine.

11. The integrative vector according to claim 7, characterized in that gene expression is under the control of the xylA promoter.

12. The integrative vector according to claim 7, characterized in that it comprises at least one temperature-sensitive origin of replication.

13. The integrative vector according to claim 7, characterized in that it comprises the temperature-sensitive origin of replication pE194ts.

14. A nucleotide sequence as shown in SEQ ID No. 1, coding for a coproporphyrinogen-III oxidase.

15. The nucleotide sequence according to claim 14, characterized in that it comprises sequences with a regulatory function which are arranged upstream and/or downstream of the region, of the hemZ gene, which codes for a coproporphyrinogen-III oxidase.

16. The nucleotide sequence according to claim 14, characterized in that it originates from Bacillus megaterium.

17. A coproporphyrinogen-III oxidase with an amino acid sequence as shown in SEQ ID No. 2.

18. A coproporphyrinogen-III oxidase with an amino acid sequence as shown in SEQ ID No. 2 encoded by a nucleotide sequence according to claim 14.

19. A vector comprising part of the nucleotide sequence as shown in SEQ ID No. 1 (hemZ) as antisense RNA (ashemZ) and, in operable linkage therewith, sequences for the induced gene expression, selection, replication and/or integration into the chromosome of the host cell.

20. The vector according to claim 19, comprising an antisense RNA (ashemZ) as shown in SEQ ID No. 3 and, in operable linkage therewith, sequences for the induced gene expression, selection, replication and/or integration into the chromosome of the host cell.

21. The vector according to claim 19, characterized in that gene expression is under the control of the xylA promoter.

22. The vector according to claim 19, characterized in that it comprises at least one temperature-sensitive origin of replication.

23. The vector according to claim 19, characterized in that it comprises the temperature-sensitive origin of replication pE194ts.

24. A method for the production of vitamin B12 by means of a culture comprising a genetically modified Bacillus megaterium strain according to claim 1, wherein the fermentation is carried out under aerobic conditions.

25. The method according to claim 24, characterized in that the expression of the hemA[KK]XCDBL operon and/or the expression of the nucleotide sequence which codes for an antisense RNA of the hemZ gene (ashemZ) is induced by the addition of xylose to the fermentation medium.

26. The method according to claim 25, characterized in that the expression of the hemA[KK]XCDBL operon as shown in SEQ ID No. 4 and/or the expression of the nucleotide sequence which codes for an antisense RNA of the hemZ gene (ashemZ) as shown in SEQ ID No. 3 is induced by the addition of xylose to the fermentation medium.

27. The method according to claim 24, characterized in that, in the exponential growth phase of the aerobically fermented cells, a transition from aerobic to anaerobic fermentation conditions takes place.

28. The method according to claim 24, characterized in that at least cobalt and/or 5-aminolavulic acid is/are added to the culture medium.

29-33. (canceled)

34. A method for the preparation of an antisense RNA (ashemZ) as shown in SEQ ID No. 3, characterized in that a nucleotide sequence according to claim 14 is used and the antisense RNA (ashemZ) is produced.

35. A method for the preparation of a vector according to claim 19 comprising introducing an antisense RNA (ashemZ) as shown in SEQ ID No. 3 into a vector.

36. A method for the preparation of a genetically modified Bacillus megaterium strain comprising a gene hemA[KK] as shown in SEQ ID No. 4 coding for a feedback-resistant glutamyl-tRNA reductase, and/or a part of the nucleotide sequence of the hemZ gene as shown in SEQ ID No. 1 (hemZ) as antisense RNA (ashemZ) comprising introducing a vector according to claim 19 into a Bacillus megaterium strain.

37. A method for the preparation of a genetically modified Bacillus megaterium strain comprising a gene hemA[KK] as shown in SEQ ID No. 4 coding for a feedback-resistant glutamyl-tRNA reductase, and/or a part of the nucleotide sequence of the hemZ gene as shown in SEQ ID No. 1 (hemZ) as antisense RNA (ashemZ) comprising introducing an integrative vector according to claim 7 into a Bacillus megaterium strain.

38. A method for the production of vitamin B12 comprising growing a genetically modified Bacillus megaterium strain according to claim 1 and recovering vitamin B12.


SEQUENCE LISTING (15 sequences)

SEQ ID NO: 1-5 - skipped (5 entries)

SEQ ID NO: 6 - DNA, 39 bp, Artificial sequence
  misc_feature (1)..(39): PCR primer forward for cloning hemA[KK]
ggggactagtcaaatgcataaaaaaattatagcagtcgg

SEQ ID NO: 7 - DNA, 33 bp, Artificial sequence
  misc_feature (1)..(33): PCR primer reverse for cloning hemA[KK]
ctggggtaccccatatcaaccattattcaatcc

SEQ ID NO: 8 - DNA, 22 bp, Artificial sequence
  misc_feature (1)..(22): PCR primer 1 for cloning hemZ
tttatattcatattccattttg

SEQ ID NO: 9 - DNA, 20 bp, Artificial sequence
  misc_feature (1)..(20): PCR primer 2 for cloning hemZ
ggtaatccaaaaataaaatc

SEQ ID NO: 10 - DNA, 32 bp, Artificial sequence
  misc_feature (1)..(32): primer forward for the amplification of antisense RNA of hemZ
gcgggatcccttgaactgagcaccttgaccgg

SEQ ID NO: 11 - DNA, 39 bp, Artificial sequence
  misc_feature (1)..(39): primer reverse for the amplification of antisense RNA of hemZ
tcgactagtcggacgtaaaaaacgttcatcttctatacc

SEQ ID NO: 12 - DNA, 198 bp, Bacillus megaterium
  gene (1)..(198): part-sequence of hemZ from the 5′ region comprising -35/-10 box,ribosomal binding site and start codon for hemZ
ccgtttgtttcctgtccgcgcattcccttgaactgagcaccttgaccggacatacgtaggttttgtaaactgattacttagatagaattgatttgaaaggtgattatattgaacatttatataaaaggtatagaagatgaacgttttttacgtccgcttcaccgaatttcagatttgttttttgaagaaagcaacgtc

SEQ ID NO: 13 - protein, 25 aa, Salmonella typhimurium
  misc_feature: HemA; First 27 amino acids of alignment
MTLLALGINHKTAPVSLRERVTFSP

SEQ ID NO: 14 - protein, 25 aa, Bacillus megaterium
  misc_feature: HemA; First 27 amino acids of alignment
MHIIAVGLNFRTAPVEIREKLSFNE

SEQ ID NO: 15 - protein, 27 aa, Bacillus megaterium
  misc_feature: HemAKK; First 27 amino acids of alignment
MHKKIIAVGLNFRTAPVEIREKLSFNE